(19) United States
(12) Patent Application Publication
(10) Pub. No.: US 2019/0359939 A1
(43) Pub. Date: Nov. 28, 2019

(54) NEW USE

(71) Applicant: DIAMYD MEDICAL AB, Stockholm (SE)

(72) Inventor: Anders ESSEN-MÖLLER, Stockholm (SE)

(21) Appl. No.: 15/752,234

(22) PCT Filed: Aug. 23, 2016
(86) PCT No.: PCT/SE2016/050786

§ 371 Date: Feb. 12, 2018

(30) Foreign Application Priority Data
Aug. 23, 2015 (SE) SE1530121-1

(51) Int. Cl. C12N 5/0784; C12N 5/0783; C12N 5/0781; C12N 5/078; C12N 5/0786; C12N 5/0789; A61K 35/17; A61K 35/15; A61K 35/19

(52) U.S. Cl. CPC C12N 5/0639; C12N 2501/998; C12N 5/0635; C12N 5/0644; C12N 5/0645; C12N 5/0647; A61K 35/17; A61K 35/15; A61K 35/19; C12N 2501/845; C12N 2500/40; C12N 2501/71; C12N 2501/73; C12N 2501/2335; C12N 2501/231; C12N 2501/24; C12N 2501/15; C12N 2500/38; C12N 2502/025; C12N 5/0636

(57) ABSTRACT

The present invention relates to a method for in vitro cultivation of hematopoietic and/or mesenchymal cells, wherein said cells are cultivated in a cultivation medium comprising gamma-aminobutyric acid (GABA) or a GABA receptor agonist, to cells obtained by such a method and therapeutic use of such cells in methods of treatment of autoimmune, inflammatory or allergic disorder. The invention further relates to pharmaceutical compositions comprising the cells and cultivation media for use in the method according to the invention.

### FIELD OF THE INVENTION

The present invention relates to the field of medicinal products and their use in therapy of disease, in particular inflammatory, autoimmune, allergic, and graft versus host disease. More specifically, the invention relates to unexpected effects of GABA on antigen presenting and other cells capable of influencing immune cells that are treated in vitro to become tolerogenic in general and or for a specific antigen and then administered to a patient to induce tolerance in general or to the specific antigens involved in the disease state.

### BACKGROUND

In an inflammatory disease state, the immune system has become reactive to an endogenous or exogenous antigen present usually at the site of inflammation (or systemic inflammation), or a temporary or chronic immune situation created by an imbalance in the system at the site. Examples of such inflammatory diseases include Type-1 diabetes, metabolic syndrome, rheumatoid arthritis, multiple scleroisis, Crohn's Disease, Irritable Bowel Disease, allergy, transplant rejection and graft versus host disease. In order to treat these inflammatory situations, systemic anti-inflammatory drugs can be used to decrease the activity of the immune cells. Another approach to treatment is to attempt to stop the immune system from reacting to the activating antigens.

Yet another approach to the treatment of such disease states is the use of tolerant or tolerogenic cells. The idea behind such a treatment is to subject the immune system to immune cells, which when activated will decrease the reactivity of the surrounding immune cells. Such an approach is proposed to be performed either with unspecific tolerogenic cells, which are proposed to decrease the immune reactivity in a general manner usually at the site of inflammation, or to be performed with antigen specific tolerant or tolerogenic cells.

Tolerogenic cells can be achieved by treatment of appropriate cell samples with agents that promote a tolerogenic character. Antigen specific tolerogenic cells can be achieved by loading e.g. tolerogenic antigen presenting cells with antigen peptides, which in turn will induce tolerance in cells of the adaptive immune system that react to that specific antigen. Antigen specific tolerant cells can be achieved by exposing tolerogenic cells loaded with antigens to adaptive immune cells such as CD4+T cells. The intreraction of tolerogenic cells and adaptive cells could also be done simultaneously with the induction of tolerogenicity.

The tolerogenicity is likely a result of an induction of the production of anitinflammatory cytokines such as TGF-beta and IL-10 by both adaptive immune cells and Antigen Presenting Cells (APCs) and a regulation of certain cell surface molecules such as CD80, CD86, CD40, PD-L1, and PD-L2 on APCs and CD28 and programmed death receptors (PDrs) on e.g. CD4+ T-cells. The interaction of cells in this milieu and cellular state will drive both naïve adaptive cells and immature antigen presenting cells to become tolerant and tolerogenic respectively for a present antigen peptide.

An example of a tolerogenic cell that is proposed to be used for the treatment of inflammatory disease is the antigen presenting dendritic cell. Such cells can be derived from e.g. blood, bone marrow, adipose tissue, chord blood or Wharton's jelly.

Administration of tolerogenic DCs has been reasonably successful in treating a number of autoimmune diseases in animal models not limited to rheumatoid arthritis (RA), multiple sclerosis (MS), primary Sjögren's syndrome, immune thrombocytopenic purpura, type 1 diabetes (T1D), uveoretinitis, and experimental autoimmune encephalomyelitis (Spanier, J A et al., Journal of Neuroimmunology, vol. 286, 15 Sep. 2015, pp. 48-58; Seungbo Yoo, Sang-Jun Ha, Immune Network 2016; 16(1):52-60 and references cited therein). In these studies the systemic administration of tolerogenic DCs was shown to prevent or ameliorate the disease or condition by modulating the immune response. Depending on the disease and how the DCs had been cultivated and stimulated the immune response was modulated differently, either through a non-specific anti-inflammatory response, by an antigen-specific shift in the Th1/Th2 balance, antigen-specific CD4+ T-cell apoptosis and/or anergy, and/or by generating a Treg response.

Similarly, tolerogenic DCs have been shown to protect against graft versus host disease in several animal models, with some proof of concept studies already in clinical trials (Moreau A. et al. Transplant Res. 2012 Sep. 28; 1(1):13; Moreau A. et al., Front Immunol. 2012 Aug. 9; 3:218; Huang Y L et al., Scand J Immunol. 2011 February; 73(2):91-101; Adorini L, et al., J Cell Biochem. 2003 Feb. 1; 88(2):227-33)

A mature DC may express high numbers of co-stimulatory proteins like CD40, CD80 (B7-CD28L) and CD86 (B7-CD28 ligand) as well as antigen presenting complexes such as MCH Class II (endocytosed antigen presentation protein complex). Immature DCs (iDCs) express low amounts of these surface molecules. iDCs do not usually activate immune cells such as Th cells (T-helper cells; CD3+CD4+ cells). iDCs are, however, unstable and can be activated in vivo into mature pro-inflammatory DCs by contact with antigens and signals frequently encountered in an active immune reaction situation. Tolerogenic DCs, on the other hand, are iDCs that have been activated in such a way that they will not only not mature into pro-inflammatory mature DCs (mDCs) in contact with inflammatory signals in vivo (i.e. they are stable), but will also produce anti-inflammatory signals and induce the maturation of anti-inflammatory T-cells (such as regulatory Th-cells) When in contact with naïve immune cells, these DCs are loaded with antigens (DCs will take up protein antigens spontaneously from the surrounding solution either in vitro or in vivo), naïve T-cells with specificity towards these antigens will be activated and induced to proliferate, and will be driven towards an anti-inflammatory phenotype.

Typically, these tolerogenic DCs (tDCs) display a surface receptor pattern like that of iDCs (low expression of CD40, CD80, CD86), and often low expression of MHC Class II, but will not as iDCs be induced to increased expression by activating signals such as LPS (or MPLA—monophosphoryl lipid A, which has the same effect as LPS on immune cells)—at least not as much.

DCs are most usually generated by the cultivation of hematopoeitic cells from bone marrow or blood (or other sources) in presence of stimulating factor GM-CSF (Witmer-Pack 1987) and mesenchymal stem cells, such as from Wharton's jelly (WJMSC). Often, a combination of GM-CSF and IL-4 is used. Tolerogenicity can be achieved by cultivating DCs in the presence of cytokines such as IL-10 or TGF-beta. A more typical procedure for generating tDCs is by exposing bone marrow derived cells to Dexamethasone (Dex). Another approach is to combine Dex with Vitamin D3 (D3) treatment, another chemical which has shown to induce tolerogenicity. A tolerogenic DC profile has also been achieved by adding certain cytokines to the cultivation media, or by cultivating cells with mesenchymal stem cells.

An example of such a procedure is the one described by Ning et. al. whereby murine bone marrow cells isolated with 10 ng/ml GM-CSF and 1 ng/ml IL-4, after which cells were cultured for 6 days in the presence of 20 ng/ml IL-10 and 20 ng/ml TGF-beta. (Ning B, Wei J, Zhang A, Gong W, Fu J, et al. (2015) Antigen-Specific Tolerogenic Dendritic Cells Ameliorate the Severity of Murine Collagen-Induced Arthritis. PLoS ONE 10(6): e0131152)

Another example is the similar generation of tDC from human blood cells described by Garcia-Gonzáles et.al. Human monocyts were isolated from buffy coats prepared from blood samples from healthy donors. Monocytes were cultured for 5 days in the presence of 500 U/ml of GM-CSF and 500 U/ml of IL-4. Medium was replaced on day 3, at which time 1 μM Dex was also added to induce tolerogenicity. 1 μg/ml MPLA was added on day 4 to activate DCs. (Garcia-Gonzalez et al., Journal of Translational Medicine 2013, 11, 128)

The tolerogenic profile of tDCs may be characterised by e.g. FACS analysis staining for the expression of surface factors such as the co-stimulatory factors CD80, CD86 and D40, antigen presenting complex MHC Class II, as well as intracellular factors such as the cytokines IL-10, IL-12, TGF-beta. Secretion of cytokines by the cells can also be analysed by ELISA or ELISpot. The successful generation of tDCs would generally be indicated, as implied earlier, by an increased presence of cells with low expression of the mentioned surface factors, and/or high expression of factors such as IL-10, TGF-beta, Programmed Death Ligand 1, Programmed Death Ligand 2, or low expression of IL-12. To characterize DCs specifically, FACS analysis is done by gating for CD11c+ monocytes.

### SUMMARY

The present inventor has unexpectedly found that addition of gamma-aminobutyric acid (GABA) to the growth medium used in cultivation of cells can produce tolerogenic cells comparable or better than what has been achieved in the prior art. The present invention thus in a first aspect relates to methods for cultivating hematopoietic and/or mesenchymal cells in a cultivation medium, wherein the cultivation medium comprises gamma-aminobutyric acid (GABA) or a GABA receptor agonist.

The invention further relates to cells obtained or obtainable by the method according to the above aspect, and to their use in therapy, in particular therapy of autoimmune, inflammatory and allergic disorders.

The invention further relates to pharmaceutical compositions comprising cells obtained by the method according to the invention. Such cells are preferably tolerogenic.

The invention further relates to a composition suitable for cultivation of mammalian cells, i.e. a mammalian cell cultivation medium, comprising GABA or a GABA receptor agonist.

The invention is as set out in the appended claims.

### BRIEF DESCRIPTION OF THE DRAWINGS

FIG. 1: Dendritic cell (CD11c+ monocytes) surface receptor expression after cultivation with various additives. Expression as indicated by the MFI of CD11+ monocyte cells stained with fluorescent antibodies towards CD40, CD80, CD86 and MHC Class II. A: comparing cells untreated (iDCs), matured with MPLA (mDCs), and cells treated with GABA, GABA and rhGAD65, and GABA and rhGAD65 matured with MPLA. B: comparing cells untreated (iDCs), matured with MPLA (mDCs), and cells treated with GABA, GABA and alhydrogel adsobed rhGAD65, and GABA and alhydrogel adsorbed rhGAD65 matured with MPLA. C: comparing cells untreated (iDCs), matured with MPLA (mDCs), and cells treated with GABA and rhGAD65 matured with MPLA, GABA and alhydrogel adsorbed rhGAD65 matured with MPLA, and cells treated with Dex and VD3 and matured with MPLA.

FIG. 2: IL-12 production of treated dendritic cells. A: FACS plots showing the intracellular IL-12 expression of CD11c+ monocytes as percent IL-12+ cells of parent population; from left to right, untreated (iDCs), matured by MPLA (mDCs), tolerogenic by Dex and VD3 and tolerogenic by GABA. B: Column diagram of percent of parent numbers of the same groups.

### DEFINITIONS

The term “tolerogenic” denotes the capacity of inducing immunologic tolerance in naïve immune cells.

The term “immunological tolerance” denotes a specific state of immune cells whereby exposure to certain specific antigens causes the cells to lower the inflammatory reaction in the surrounding mileu, while under other conditions the antigen is capable of inducing an inflammatory reaction. Cells having this property are said to be “tolerant” or “of tolerant character”. Examples of cells of such a property are regulatory T-cells.

A “GABA receptor agonist” refers generally, as used herein, to a compound that directly or indirectly activates a GABA-receptor, such as the GABA-A receptor, GABA-B receptor, or GABA-C receptor, relative to the activity of the receptor in the absence of the compound. Some examples of GABA-receptor agonists are muscimol, vigabatrin and baclofen.

### DETAILED DESCRIPTION

The present inventor has surprisingly found that cultivation of hematopoietic or mesenchymal cells in cultivation medium comprising GABA or a GABA receptor agonist induces a surface protein expression pattern indicative of tolerogenicity in said cells. Accordingly, the present invention relates to methods and media formulations for using GABA and GABA receptor agonists for producing tolerogenic cells, and the use of such tolerogenic cells in therapeutic methods.

In our experiments with DCs from murine bone marrow (NOD mice), cultivation in the presence of 100 μM GABA produces a surface protein expression profile similar to that of iDCs, indicative of a tolerogenic profile. When loaded with antigen GAD65, surface expression remains the same. Upon stabilization with MPLA the expression increases slightly but is still much lower than mDCs, which is indicative of stabilized and activated tolerogenic cells

When compared to a common protocol of producing stable tDCs using Dexamethasone, Vitamin D and MPLA, tolerogenic DCs produced by cultivation with 100 μM GABA and stabilization with MPLA display a comparable or better tolerogenic expression pattern.

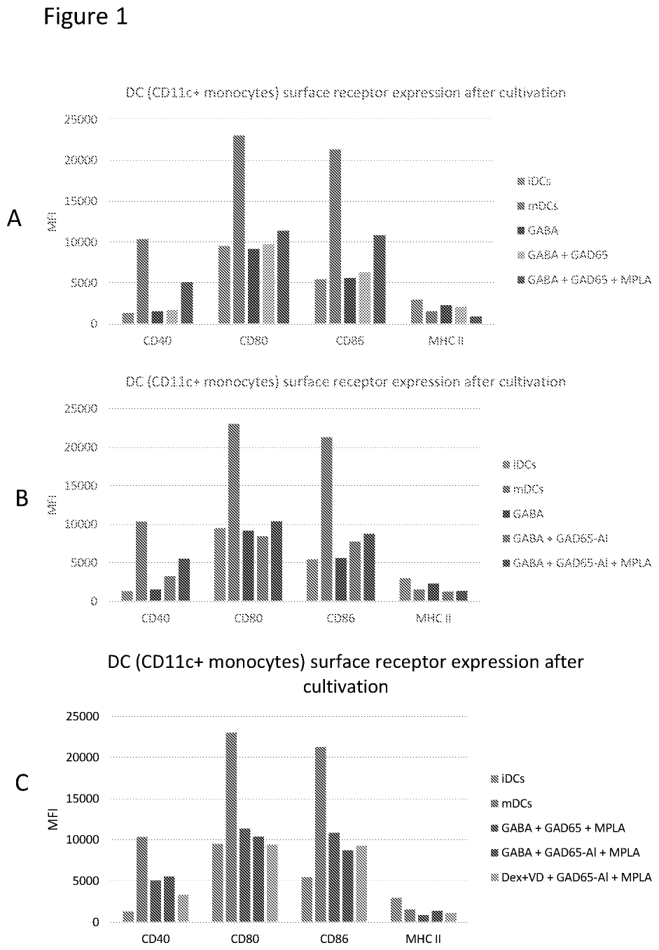

FIG. 1 (A, B and C) show the surface expression pattern of CD80, CD86, CD40 and MCH Class II proteins on murine DCs treated according to different protocols. Where iDCs (untreated cells) display a low expression of said surface proteins, proinflammatory mDCs activated by MPLS (or LPS) display a high expression, and tDCs display a relatively low expression.

FIG. 1A shows that DCs treated with GABA display an expression pattern more similar to iDCs and indicative of tDCs. When both GABA and antigen, in this case rhGAD65, is added to the cells, the pattern remains indicative of tDCs. When MPLA is added in addition to GABA and rhGAD65, an addition that otherwise changes iDCs into cells with an mDC profile, the expression pattern still remains almost the same (still indicative of tDCs).

FIG. 1B shows the same results as 1A but alhydrogel adsorbed rhGAD65 has been added instead of pure rhGAD65. The same pattern is seen, indicating that cells that have been treated with GABA and alhrydrogel adsorbed rhGAD65 keep a low surface molecule expression even when MPLA activator is added. The MPLA even seems to have less effect on these cells.

FIG. 1C shows a comparison of the expression profiles of iDCs and mDCs with cells treated with GABA and rhGAD65 activated by MPLA, GABA and alhydrogel adsorbed rhGAD65 activated by MPLA, and DCs treated with Dex, VD3 and alhydrogel adsorbed rhGAD65 activated with MPLA (a common tolerogenic treatment). The GABA treated DCs display a profile similar to those treated with Dex and VD3. The CD80 expression is even lower for GABA treated cells.

DCs can also be characterized by staining of the cytokines they produce, either by intracellular staining followed by FACS analysis, or by ELISpot analysis (counting cells that make cytokines), or by quantifying the cytokine release of the cells (e.g. ELISA or Luminex etc).

Tolerogenic DCs display lower IL-12, IL-23 and TNFα release levels compared to mature DCs (Garcia-Gonzalez et al. Journal of Translational Medicine 2013, 11:128). Our experiments show that DCs treated with 100 μM GABA display an intracellular IL-12 cytokine production similar to that of tolerogenic DCs produced by cultivation with Dexomethazone and Vitamin, and very different from mDCs.

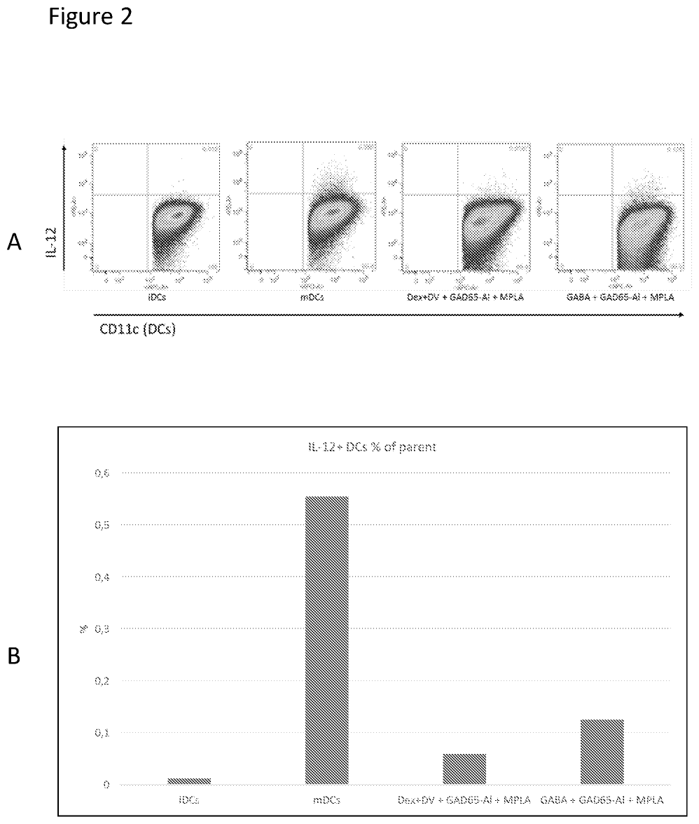

In FIG. 2A, FACS plots of the intracellular expression of IL-12 of untreated CD11c+ DCs (iDCs), proinflammatory matured DCs (mDCs), DCs treated with Dex, VD3 and alhydrogel adsorbed rhGAD65 activated by MPLA, and DCs treated with GABA and alhydrogel adsorbed rhGAD65 activated by MPLA are shown. IL-12 is a proinflammatory cytokine, and low production is indicative of tolerogenic DCs. The IL-12+ cells are shown in the upper left quadrant of the plots.

FIG. 2B shows the numerical representation of the FACS plots. i.e. the percentage of cells (CD11c+ monocytes) that are IL-12 positive. iDCs generally display a low expression of IL-12, which is to be expected, and a large number of the mDCs are IL-12+. Tolerogenically treated cells on the other hand generally have very low expression of IL-12. Both cells treated with Dex and VD3 and those treated with GABA, even though they have been activated with MPLA, show very low numbers of IL-12+ cells.

Together, this data indicates that treatment of DCs with 100 μM GABA can generate cells of a tolerogenic character.

Such antigen loaded tolerogenic DCs, or antigen-specific Th cells made tolerant by in vitro exposure to tolerogenic DCs, could be introduced into a patient suffering from an inflammatory condition fuelled by (among other factors) an immune reaction to that specific antigen, in order to counteract the inflammatory process. Such a treatment would alleviate the symptoms, or possibly rebalance the immune system and cure the disease or condition.

Thus, the invention in a first aspect relates to a method for in vitro cultivation of hematopoietic or mesenchymal cells, wherein said cells are cultivated in a cultivation medium comprising gamma-aminobutyric acid (GABA) or a GABA receptor agonist. The hematopoietic or mesenchymal cells may be dendritic cells, hematopoietic stem cells, antigen-presenting cells, monocytes, macrophages, neutrophils, platelets, T-lymphocytes, B lymphocytes, and mesenchymal stem cells such as from Wharton's jelly.

The cultivation medium may comprise GABA or GABA receptor agonist in a concentration of 10-1000 μmole/liter, such as 50-500 μmole/liter. The concentration of GABA or GABA receptor agonist is preferably around 100 μmole/liter.

The cells may be cultivated for a number of days, such as 4, 5, 6, 7, or 8 days, preferably 6 days, without GABA or GABA receptor agonist being present in the cultivation medium. After GABA has been added to the cultivation medium, the cells may be cultivated for a further 12-72 hours, such as about 24 to 60 hours, or about 48 hours.

The cultivation medium may further comprise at least one of CRAMP, Zebularine, indoleamine 2,3-dioxygenase-1 (IDO1), cytidine, a cytidine analogue, kynureninase, IL-10, IL-35, IFN-gamma, TGF-beta-1, a vitamin D3 compound, or any derivative thereof, and mesenchymal stem cells such as from Wharton's jelly. The cultivation medium may also further comprise at least one of a CTLA4-inhibitor, abatacept, a TNF-alpha inhibitor, and alefacept

In a further embodiment, the cultivated cells are exposed to at least one endogenous or exogenous antigen. Examples of endogenous antigens are proinsulin, insulin, IA-2, ZnT8, GAD, GAD65, GAD67, transglutaminase, myelin basic protein, MOG, collagen-2, thyroglobulin, thyroid peroxidase, MHC I, MHC II, or any derivative or fragment thereof. Examples of exogenous antigens are food allergens (such as gliadin, peanut allergens, shrimp allergens, egg allergens), animal allergens (such as allergens from cat, dog, horse), mite allergens, and plant allergens (such as tree and grass pollen allergens), or any fragment or derivative thereof, or transplanted cells and/or tissue.

The invention further relates to cells obtained and/or obtainable by the method according to the invention, as described above. Such cells are are preferably tolerogenic and capable of inducing maturation of naïve T-cells into mature T-cells of tolerant character having specificity to at least one endogenous or exogenous antigen.

The invention also relates to methods of treatment of an autoimmune, inflammatory or allergic disorder comprising administering cells obtained or obtainable by the method according to the invention to a patient suffering from said disorder, and to the use of such cells in such methods.

Examples of disorders that are amenable to treatment using cells obtained or obtainable by the method according to the invention are asthma, allergy, acute sinusitis, chronic sinusitis, Addison's disease, autoimmune hepatitis, Behcet's disease, coeliac disease, chronic active hepatitis, contact dermatitis, ulcerative colitis, Crohn's disease, inflammatory bowel disease (IBD), ulcerative colitis, systemic lupus erythematosus (SLE), Amyotrophic Lateral Sclerosis (ALS), Psoriasis, Psoriasis Arthritis, Juvenile Arthritis, Churg-Strauss Syndrome, idiopathic thrombocytopenic purpura, dermatomyositis, eczema, Goodpasture's syndrome, Guillian-Barre syndrome, Hashimoto's encephalitis, Hashimoto's thyroiditis, Graves disease, juvenile diabetes, juvenile onset (Type I) diabetes mellitus, latent autoimmune diabetes in the adult (LADA), type 2 diabetes, multiple sclerosis (MS), spino-optical MS, opsoclonus myoclonus syndrome (OMS), paraneoplastic cerebellar degeneration, pernicious anemia (anemia perniciosa), polymyositis, primary biliary cirrhosis, rheumatoid arthritis, sarcoidosis, scleroderma, lupus erythematosus, temporal arteritis/giant cell arteritis, primary Sjögren's syndrome, immune thrombocytopenic purpura, rejection of transplant and graft versus host disease.

The cells used in the method of treatment may originate from the subject to be treated. In this embodiment, hematopoietic or mesenchymal cells are removed from the subject according to standard protocols as known in the art, cultivated according to the present invention, and then returned to the subject.

The cells used in the method of treatment may also originate from a donor different from the subject to be treated. In this embodiment, hematopoietic or mesenchymal cells are removed from the donor according to standard protocols as known in the art, cultivated according to the present invention, and then administered to the subject.

The form and manner of administration of the cells according to the invention are determined by the treating medical practitioner, as is the number or dose of cells. Therapeutic methods using tolerogenic cells are known in the art and have been studied in clinical trials (Spanier, J A et al., Journal of Neuroimmunology, vol. 286, 15 Sep. 2015, pp. 48-58; Seungbo Yoo, Sang-Jun Ha, Immune Network 2016; 16(1):52-60 and references cited therein).

The invention further relates to a pharmaceutical composition comprising cells obtained or obtainable by the method according to the invention. The pharmaceutrical composition may optionally comprise additional pharmaceutically acceptable solvents, buffers, excipients.

The invention also relates to media for cultivation of mammalian cells in vitro, which media comprise GABA or GABA receptor agonist. Mammalian cell cultivation media in general are well-known in the art (see e.g. Sato and Kan, Media for Culture of Mammalian Cells, in Current Protocols in Cell Biology, 2001, John Wiley & Sons, Inc.). The cultivation media may also further comprise at least one of CRAMP, Zebularine, indoleamine 2,3-dioxygenase-1 (IDO1), cytidine, a cytidine analogue, kynureninase, IL-10, IL-35, IFN-gamma, TGF-beta-1, a vitamin D3 compound, or any derivative thereof, and mesenchymal stem cells, such as from Wharton's jelly. The cultivation media may also further comprise at least one of at least one of a CTLA4-inhibitor, abatacept, a TNF-alpha inhibitor, alefacept.

### EXAMPLES

The examples below serve to illustrate the invention and shall not be construed as limiting the scope of the invention, which is that of the appended claims.

### Example 1

Preparation of Dendritic Cells Derived from Mouse Bone Marrow

6 week old C57BL/6 and BALB/c mice are purchased from a supplier. After an acclimatisation period of 1 week, the mice are used for the experiments.

Dendritic cells obtained from mouse bone marrow are used. In order to secure a large amount of the number of the dendritic cells, Inaba's culture method (Inaba et al., J. Exp. Med. 175:1157, 1992), which is a representative method of culturing a dendritic cell, is modified and applied to the dendritic cells. The number of the dendritic cells obtained from bone marrow cells will be at about 10 to 15% of bone marrow cells in an initial culture. A tibia and a femur of a mouse aged 6 to 11 weeks are extracted, and a RPMI1640 medium is added to bone marrow of the tibia and the femur to obtain bone marrow cells therefrom. Then, hypotonic ammonium chloride-potassium (ACK) lysing buffer is used for the lysis of red blood cells. The bone marrow cells are cultured in an RPMI1640 medium containing about 10 to 20 ng/ml recombinant mouse GMCSF, about 5 to 10 ng/ml recombinant mouse IL4, 5 to 10% heat inactivated fetal bovine serum, L-glutamine, 25 mM of HEPES, penicillin, and streptomycin under conditions of 5% CO₂ and a temperature of 37° C., thereby inducing differentiation into dendritic cells. The medium is replaced every 2 days with a medium containing the recombinant cytokines. After 2 and 4 days of the culture, granulocytes and lymphocytes that are floating on the culture dish are removed. After 6 days of the culture, dendritic cells that are differentiated from precursor cells attached on the bottom of the culture dish and that have characteristic protrusions in suspension are used in the experiments. In order to confirm purity of the prepared dendritic cells, the dendritic cells are stained with FITC conjugated anti-CD11c antibody against CD11c molecule that is highly expressed on the surface. Then, it is confirmed by flow cytometry that the ratio of the CD11c-positive dendritic cells is 85% or more.

### Example 2

Identification of Immaturity of Dendritic Cells Based on Surface Phenotypes.

Accordingly, antigens are exposed to unsensitized lymphocytes so as to induce proliferation and differentiation of the lymphocytes.

About 1,000,000 to 2,000,000 of the dendritic cells are subjected to flotation in about 2 to 4 ml of the medium prepared above to be placed in two 6-well plates. Groups of the dendritic cells are subject to the addition of GABA or GABA receptor agonist, and optionally at least one of Zebularine, IL35, vitamin D3 or combinations thereof and Mouse Serum Albumin or endotoxin (LPS, E. coli serotype O55:B5) in a range of about 0.01 to 1 mg/ml, as positive control.

After 12-48 hours the dendritic cells are treated with recombinant human GAD65 and/or proinsulin in a concentration in a range of about 0.1 to 5 ug/ml.

After 12 to 48 hours of the culture, fluorescent labeled antibodies with respect to surface molecules of the dendritic cells (e.g., antiCD11 . . . ) are used to stain the dendritic cells for 20 minutes to 1 hour at a temperature of 4° C. Then, the results are obtained by flow cytometry. It is observed that the amount of maturation markers on the surface of the dendritic cells is decreased by GABA treatment compared to LPS or mouse serum albumin treatment. That is, the increased expression of CD40, CD80, CD86, and MHC class II in the GABA treated dendritic cells is found to be less than those treated by LPS or Mouse Serum Albumin.

### Example 3

Mouse bone marrow-derived dendritic cells (BMDCs) were generated from bone marrow cells (femura) of 8-week-old NOD mouse females in complete RPMI 1640 medium (Lonza, Basel Switzerland) in the presence of GM-CSF (20 ng/ml) and IL-4 (4.5 ng/ml, PeproTech) for 6 days. Fresh medium was added on day 3 and comparator tDCs were induced by adding dexamethasone (1 μM) and vitamin D3 (0.5 ng/ml) on day 6, while mDCs were generated without tolerogenic factors, and iDCs were untreated for the entire cultivation period. Similarly for GABA testing, GABA was added on day 6 in at a concentration of 100 μM. Exposure to antigen was achieved by adding GAD65 (either pure GAD65 protein or alum adsorbed GAD65) to the cultivation mixture on day 6 along with tolerizing agents at a concentration of 5 μg/ml. In some cultivations final maturation (mDCs, tDCs and GABA treated cells) was achieved at day 7 by activating DCs with 2 μg/ml VacciGrade MPLA (monophosphoryl lipid A, InvivoGen) for 24 h. Cells were harvested on day 8.

Mouse DCs were characterized by flowcytometry using following mAbs purchased from eBioscience; anti-CD11c-APC (clone N418), anti-CD40-PerCP-eFluor710 (clone 1C10), anti-CD80-FITC (clone 16-10A1), anti-CD86-PE (clone GL1), and anti-MHC II (I-A/I-E) (clone MS/114.15.2). Cells were stained with fluorochrome-conjugated antibodies against surface molecules and data were acquired by LSR II flow cytometers (BD Biosciences) and analyzed using FlowJo software (Tree Star).

1. A method for in vitro cultivation of hematopoietic and/or mesenchymal cells, comprising cultivating said cells in a cultivation medium comprising gamma-aminobutyric acid (GABA) or a GABA receptor agonist.

2. The method according to claim 1, wherein the hematopoietic and/or mesenchymal cells are selected from the group consisting of dendritic cells, hematopoietic stem cells, antigen-presenting cells, monocytes, macrophages, neutrophils, platelets, T-lymphocytes and B lymphocytes.

3. The method according to claim 1, wherein the cultivation medium comprises GABA or GABA receptor agonist in a concentration of 10-1000 μmole/liter.

4. The method according to claim 1, wherein the cultivation medium further comprises at least one of CRAMP, Zebularine, indoleamine 2,3-dioxygenase-1 (IDO1), cytidine, a cytidine analogue, kynureninase, IL-10, IL-35, IFN-gamma, TGF-beta-1, a vitamin D3 compound, or any derivative thereof, and mesenchymal stem cells, such as from Wharton's jelly.

5. The method according to claim 1, wherein the hematopoietic and/or mesenchymal cells are induced to be tolerogenic.

6. The method according to claim 1, further comprising exposing the hematopoietic and/or mesenchymal cells for at least one endogenous or exogenous antigen.

7. The method according to claim 1, wherein the at least one endogenous antigen is selected from the group consisting of proinsulin, insulin, IA-2, ZnT8, GAD, GAD65, GAD67, transglutaminase, myelin basic protein, MOG, collagen-2, thyroglobulin, thyroid peroxidase, MHC I, MHC II, or any derivative or fragment thereof.

8. The method according to claim 1, wherein at least one exogenous antigen is selected from the group consisting of food allergens (such as gliadin, peanut allergens, shrimp allergens, egg allergens), animal allergens (such as allergens from cat, dog, horse), mite allergens, and plant allergens (such as tree and grass pollen allergens), or any derivative or fragment thereof, or transplanted cells and/or tissue.

9. The method according to claim 1, wherein the cultivation medium further comprises at least one of a CTLA4-inhibitor, abatacept, a TNF-alpha inhibitor, alefacept.

10. Hematopoietic and/or mesenchymal cells obtained by a method according to claim 1.

11. The hematopoietic and/or mesenchymal cells according to claim 10, which are tolerogenic and capable of inducing maturation of naïve T-cells into mature T-cells of tolerant character.

12. The hematopoietic and/or mesenchymal cells according to claim 10, which are tolerogenic and capable of inducing maturation of naïve T-cells into mature T-cells of tolerant character having specificity to at least one endogenous or exogenous antigen.

13. A method of treating a subject for an autoimmune, inflammatory or allergic disorder, comprising administering hematopoietic and/or mesenchymal cells according to claim 10 to the subject.

14. The method of claim 13, wherein said disorder is selected from asthma, allergy, acute sinusitis, chronic sinusitis, Addison's disease, autoimmune hepatitis, Behcet's disease, coeliac disease, chronic active hepatitis, contact dermatitis, ulcerative colitis, Crohn's disease, inflammatory bowel disease (IBD), ulcerative colitis, systemic lupus erythematosus (SLE), Amyotrophic Lateral Sclerosis (ALS), Psoriasis, Psoriasis Arthritis, Juvenile Arthritis, Churg-Strauss Syndrome, idiopathic thrombocytopenic purpura, dermatomyositis, eczema, Goodpasture's syndrome, Guillian-Barre syndrome, Hashimoto's encephalitis, Hashimoto's thyroiditis, Graves disease, juvenile diabetes, juvenile onset (Type I) diabetes mellitus, latent autoimmune diabetes in the adult (LADA), type 2 diabetes, multiple sclerosis (MS), spino-optical MS, opsoclonus myoclonus syndrome (OMS), paraneoplastic cerebellar degeneration, pernicious anemia (anemia perniciosa), polymyositis, primary biliary cirrhosis, rheumatoid arthritis, sarcoidosis, scleroderma, lupus erythematosus, temporal arteritis/giant cell arteritis, primary Sjögren's syndrome, immune thrombocytopenic purpura, transplant rejection, and graft versus host disease.

15. The method of claim 13, wherein the method comprises returning said cells to an original donor suffering from an autoimmune, inflammatory or allergic disorder.

16. The method of claim 13, wherein the method comprises administering said cells to a subject suffering from an autoimmune, inflammatory or allergic disorder, wherein the cells originate from a donor different from the subject to be treated.

17. A pharmaceutical composition comprising cells obtained by the method according to claim 1.

18. A mammalian cell cultivation medium comprising gamma-aminobutyric acid (GABA) or a GABA receptor agonist.

19. The mammalian cell cultivation medium according to claim 18, wherein the medium comprises GABA or GABA receptor agonist in a concentration of 10-1000 μmole/liter.

20. The mammalian cell cultivation medium according to claim 18, wherein the medium further comprises at least one of CRAMP, Zebularine, indoleamine 2,3-dioxygenase-1 (IDO1), cytidine, a cytidine analogue, kynureninase, IL-10, IL-35, IFN-gamma, TGF-beta-1, a vitamin D3 compound, or any derivative thereof, and mesenchymal stem cells, such as from Wharton's jelly, and/or at least one of at least one of a CTLA4-inhibitor, abatacept, a TNF-alpha inhibitor, alefacept.
